(19) United States
(12) Patent Application Publication
(10) Pub. No.: US 2022/0317120 A1
(43) Pub. Date: Oct. 6, 2022

(54) USE OF PROTEINS PD-1 AND CD38 AS MARKERS OF AN ACTIVE AUTO-IMMUNE PATHOLOGY

(71) Applicants: UNIVERSITÉ DE NANTES, Nantes (FR); CENTRE HOSPITALIER UNIVERSITAIRE DE NANTES, Nantes (FR); INSERM - INSTITUT NATIONAL DE LA SANTÉ ET DE LA RECHERCHE MÉDICALE, Paris (FR)

(72) Inventors: Amédée RENAND, Sainte-Luce-sur-Loire (FR); Sophie CONCHON, Nantes (FR); Jérôme GOURNAY, Nantes (FR); Hélène AUBLE, Nantes (FR); Sarah HABES, Nantes (FR)

(21) Appl. No.: 17/608,908

(22) PCT Filed: May 19, 2020
(86) PCT No.: PCT/EP2020/063886

§ 371 Date: Nov. 4, 2021

(30) Foreign Application Priority Data
May 22, 2019 (FR) 1905358

(51) Int. Cl. G01N 33/564; G01N 33/576

(52) U.S. Cl. CPC G01N 33/564; G01N 33/5767; G01N 2800/56

(57) ABSTRACT

The present invention relates to a method for diagnosis, prediction and/or prognosis of an active autoimmune pathology in a subject, comprising detecting the co-expression of PD-1 and CD38 proteins at the surface of T lymphocytes in a biological sample from the subject.

The present invention also relates to a use of the pool of PD-1 (Programmed cell death 1) and CD38 protein as biomarkers for diagnosis, prediction and/or prognosis of an active autoimmune pathology in a subject.

The present invention further relates to a test device for detecting the co-expression of PD-1 and CD38 in a sample from a subject, comprising:
(i) optionally means for obtaining a sample from the subject, and
(ii) means for detecting the co-expression of PD-1 and CD38 at the surface of the T lymphocytes in said sample, and
(iii) means for determining the frequency of co-expression of PD-1 and CD38 in the sample

### TECHNICAL AREA

The present invention relates to a method for diagnosis, prediction and/or prognosis of an active auto-immune pathology in a subject, comprising measuring the amounts of certain proteins in a biological sample from the subject, as well as certain uses of these proteins.

In the description below, references in square brackets ([ ]) refer to the list of references at the end of the text.

STATE OF THE ART

Auto-immune hepatitis (AIH) is a chronic liver disease whose immunological mechanisms remain unknown. It is a rare disease with an incidence of 1.1 to 1.9 cases per 100,000 people per year in Europe, characterized by an attack on the liver parenchyma by cells of the lymphoid immune system (Liberal et al.: Cutting edge issues in autoimmune hepatitis. J. Autoimmun. (2016) 75, 6-19 ([1])).

The diagnosis of AIH is often long in coming. As with other autoimmune liver diseases, it is based on the elimination of other causes, on not very specific biological and biochemical blood tests (elevated transaminases, absence of viral hepatitis, presence of autoantibodies, and hyper gammaglobulinemia), and on the histological reading of liver biopsies. In other cases of hepatic or non-hepatic auto-immune disorders, the diagnosis is based on symptoms, the presence or not of autoantibodies, and the histological reading of the affected tissue. Monitoring of the response to treatment is usually based on improvement of symptoms, regulation of biological parameters and possibly histological reading of affected tissue.

The treatment combining corticosteroids and azathioprine, unchanged for more than 30 years (Manns et al.: Diagnosis and management of autoimmune hepatitis. Hepatol. Baltim. Md 51, 2010, 2193-2213 ([2])), aims to control inflammation to avoid progression to liver fibrosis. This non-selective immunosuppression is sufficient to induce a complete remission in 70% of patients in the first year. European guidelines recommend attempting to stop azathioprine after 2 years of complete remission to limit the adverse effects of this in the long term (European Association for the Study of the Liver (2015). EASL Clinical Practice Guidelines: Autoimmune hepatitis. J. Hepatol. 63, 971-1004 ([3])); however, the risk of relapse is high and affecting 60 to 80% of patients within six to twelve months of stopping treatment (Manns et al., 2010 ([2])). The state of complete clinical remission is currently based on biological and biochemical tests, including normalization of blood transaminases and IgG, which do not predict the risk of relapse.

A better understanding/characterization of the immune response in AIH would be useful to predict the risk of relapse, but also to identify the immunological protagonists involved in the pathogenesis of this disease.

CD4 T lymphocytes (CD4 TL) and CD8 TL have been described as strongly involved in the attack of the liver parenchyma in AIH. They are largely represented in the level of the portal and intra-lobular inflammatory infiltrate. However, the relationship between the immunological players involved and the disease course is not yet known (Liberal et al., 2016 ([1])).

Recently, blood lymphocyte alterations have been demonstrated at diagnosis, which persist during the remission phase under treatment (Renand et al.,: Immune Alterations in Patients With Type 1 Autoimmune Hepatitis Persist Upon Standard

Immunosuppressive Treatment. Hepatol. Commun. (2018) 2, 968-981 ([4])). However, some of these lymphocyte alterations were found in patients with non-alcoholic steatohepatitis (NASH), and thus seemed to reflect nonspecific liver inflammation (Böttcher et al: MAIT cells are chronically activated in patients with autoimmune liver disease and promote pro-fibrogenic hepatic stellate cell activation. Hepatol. Baltim. Md, 2018 ([5]); Jeffery et al: Biliary epithelium and liver B cells exposed to bacteria activate intrahepatic MAIT cells through MRI. J. Hepatol. 2016; 64, 1118-1127 ([6]); Oo et al: CXCR3-dependent recruitment and CCR6-mediated positioning of Th-17 cells in the inflamed liver. J. Hepatol. 57, 1044-1051, 2012 ([7])). In addition, cases of non-viral liver inflammation (such as NASH) with signs of autoimmunity (presence of blood autoantibodies in 10% of NASH patients) have been reported, but no specific treatment is currently available.

Thus, there is a real need to identify a specific signature of hepatic autoimmunity, in particular to improve diagnosis or clinical and therapeutic management.

### DESCRIPTION OF THE INVENTION

After extensive research, the inventors succeeded in solving this technical problem.

Surprisingly, the inventors have identified a specific signature of hepatic autoimmune disease.

The inventors have demonstrated, by flow cytometry on blood T lymphocytes (CD3+), that the detection of PD-1 (Programmed cell death 1) and CD38 markers, possibly in association with CD3, CD4, CD8, CD45RA, CXCR5, CD127 and CD27 markers, highlights a hepatic autoimmune signature. This autoimmune signature advantageously allows to discriminate patients with active autoimmune hepatitis (AIH) from patients with non-autoimmune liver inflammation (NASH).

The advantages of the invention are numerous.

For example, the invention can allow to reduce the diagnosis time by providing new information complementary to that conventionally obtained.

The invention also has the advantage of providing a means for a better orientation and/or a confirmation of the diagnosis, especially in atypical cases (seronegative form, without autoantibodies) of AIH.

The invention may also make it possible to evaluate autoimmune activity in patients. A better management of the patients can thus be advantageously envisaged, with for example an adjustment or not of the proposed treatment.

Advantageously, the invention is applicable to other hepatic and non-hepatic autoimmune disorders which face the same problem.

In addition, the invention advantageously makes it possible to identify among patients with hepatic inflammation a subgroup of patients with associated hepatic autoimmunity. An improvement of their clinical and therapeutic management is thus advantageously envisaged.

Advantageously, the invention allows a simplified assessment of autoimmune activity in a patient, since it can be performed by detecting an autoimmune signature according to the invention in the patient's blood, for example by means of a simple blood test.

The invention also makes it possible, in an advantageous manner, to measure the activity of T lymphocytes in the blood, which are the main players in autoimmunity. This method is less invasive for the patient than a biopsy, and provides added value to already known biological tests. In addition, the direct measurement of the activity of the protagonists of the pathology in the blood provides, in an advantageous way, a better sensitivity and possibly a complement in the case of atypical diagnosis and follow-up, as for example in the seronegative forms or with weak lymphocyte infiltrate of the tissue.

Thus, a first object of the invention relates to a method of diagnosis, prediction and/or prognosis of an active autoimmune pathology in a subject, comprising detecting the co-expression of the proteins PD-1 and CD38 at the surface of T lymphocytes in a biological sample of the subject.

According to the invention, the active autoimmune pathology may be any known autoimmune pathology. In particular, it may be an autoimmune pathology selected from autoimmune hepatitis, primary biliary cholangitis, primary sclerosing cholangitis, type 1 diabetes, multiple sclerosis, rheumatoid arthritis, lupus, and vasculitis or immunological toxicity related to immunotherapies in cancer treatment. Preferentially, the active autoimmune pathology is autoimmune hepatitis. For example, the subject in whom diagnosis, prediction and/or prognosis is sought may be a patient with liver inflammation.

By “diagnosis”, in the sense of the present invention, is meant the demonstration of autoimmunity in patients. This demonstration can advantageously make it possible to deduce or confirm a patient's disease caused by AIH, or to demonstrate an autoimmune disease in the context of a predominantly non-autoimmune pathology. For example, the diagnosis can be made on atypical forms of AIH, for example a seronegative form, that is without autoantibodies, and/or with little or no lymphocytic infiltrate of the liver tissue. In another example, the diagnosis may be a discrimination of patients with autoimmune hepatitis from patients with non-alcoholic steatohepatitis (NASH), or a demonstration of hepatic autoimmunity in patients with NASH.

By “prediction”, in the sense of the present invention, is meant the anticipation of the progression of the disease in a subject during follow-up over time. It may in particular be a prediction of a risk, for example of the risk of developing an autoimmune pathology in a subject, or of the risk of relapse of a patient suffering from an autoimmune pathology, in particular following the cessation or adjustment of his or her treatment, or of an immun-related adverse event (IRAEs), for example during the use of antibodies targeting checkpoint inhibitors in the context of anti-cancer immunotherapy.

By “prognosis”, in the sense of the present invention, is meant predicting the chances of survival of a patient with an active autoimmune pathology in a subject.

For example, the method for prognosis of the invention may allow to assess the survival rate, where survival rate indicates the percentage of individuals who have participated in a study and are alive for a given period of time, typically 5 to 10 years after diagnosis of an active autoimmune pathology. Generally, a good prognosis, as defined in the invention, is associated with a median survival of more than 5 years, whereas a poor prognosis is associated with a median survival of less than 5 years.

By “detection of protein co-expression”, in the sense of the present invention, is meant any detection of the joint expression of PD-1 and CD38 proteins at the surface of each T lymphocyte present in the biological sample. It is therefore the frequency of joint expression of PD-1 and CD38 proteins at the surface of each T lymphocyte present in the biological sample that is measured. The detection can be, for example, a comparison between the level of the frequency of joint expression of these proteins in a biological sample of a subject, and a reference sample or value (also called “threshold value” or “normal value”) allowing a proven diagnosis, prediction and/or prognosis of the active autoimmune pathology. Indeed, the reference value represents the frequency of joint expression of these proteins in the population without the desired pathology. The reference value can be defined on the basis of measurements obtained on a sample of healthy subjects, that is not presenting the pathology of interest. These may be, for example, subjects who do not have viral hepatitis, signs of autoimmunity in the liver and/or chronic disease except, possibly, liver inflammation due to dysmetabolic disorder (also called “fatty liver” or “soda disease”). Thus, it is understood that the “healthy subjects” for defining the reference value may be patients with NASH (or without NASH), but without viral hepatitis, signs of autoimmunity in the liver and/or other chronic conditions apart from liver inflammation due to dysmetabolic disturbance. The reference value can be defined for example according to the protocol described in example 2 below. The reference value can be established on a significant number of healthy subjects, for example at least 100 healthy subjects. Advantageously, the threshold value may be defined as greater than a frequency of TLCD3+ co-expressing PD1 and CD38 of 2.77 percent. Thus, a frequency above 2.77 percent will be characteristic of hepatic autoimmunity. Detection can be performed by any method known to the person skilled in the art. These may be, for example, a method selected from flow or mass cytometry, an immunoassay technology, such as direct ELISA, indirect ELISA, sandwich ELISA, competitive ELISA, multiplex ELISA, radioimmunoassay (RIA) or ELISPOT technology, a mass spectrometry analysis method, a chromatography method, a qPCR method, and a combination of at least two of these methods. Thus, in one embodiment, the detection of an autoimmune signature can be performed in the blood, for example, whole blood, of the patient by a simple blood draw followed by a simplified test consisting of incubating the blood cells with a mixture of specific fluorescent antibodies, followed by analysis on a flow cytometer.

PD-1 and CD38 proteins can be detected at the surface of any cell known to express them on their surface. This may include, for example, CD3 T lymphocytes and/or CD8 T lymphocytes and/or CD4 T lymphocytes.

The biological sample used may be any biological sample commonly used for biological analysis. For example, it may be a blood sample, for example, a blood fraction, or alternatively a whole blood sample, or a biopsy fraction, for example. liver, tissue.

The method of the invention may include any or all of the steps of:
(i) detecting the co-expression of PD-1 and CD38, at the surface of T lymphocytes, in said sample and determining the frequency of co-expression of PD-1 and CD38;
(ii) comparing the frequency of co-expression of PD-1 and CD38 determined in (i) with a reference value of the frequency of co-expression of PD-1 and CD38, said reference value representing a proven diagnosis, prediction and/or prognosis of the active autoimmune pathology;
(iii) testing for the presence or absence of a deviation of the frequency of co-expression of PD-1 and CD38 determined in (ii) from the reference value;
(iv) attribute the presence or absence of a deviation to a diagnosis, prediction and/or prognosis of the subject's active autoimmune pathology.

Advantageously, a frequency of co-expression of PD-1 and CD38 in said sample above a reference value indicates that the subject has the active autoimmune pathology or presents a risk of relapse of the active autoimmune pathology.

In addition to the proteins PD-1 and CD38, according to the invention, it is possible to detect the co-expression of PD-1 and CD38 with at least one other biological marker selected from the group consisting of CD3, CD4, CD8, CD45RA, CXCR5, CD127 and CD27. This may be one marker, or two markers, or three markers, or four markers, or five markers, or six markers, or seven markers. This detection of at least one additional marker may be performed concurrently with the detection of the co-expression of PD-1 and CD38, or subsequently to the step of detecting the co-expression of PD-1 and CD38. For example, co-expression of biomarker associations selected from PD-1/CD38/CD3, PD-1/CD38/CD3/CD4/CD8/CD45RA, PD-1/CD38/CD3/CD4/CD8/CD45RA/CD127, PD-1/CD38/CD3/CD4/CD8/CD45RA/CXCR5/CD127, and PD-1/CD38/CD3/CD4/CD8/CD45RA/CXCR5/CD127/CD27 may be detected. Advantageously, the analysis of the co-expression of PD-1 and CD38 at the surface of CD3 (CD3+) TLs can be performed in association with the detection of the frequency of lymphocyte populations defined by the phenotypes below:
a) CD45RA (study of the CD3+CD45RA-PD-1+CD38+subpopulation);
b) CD45RA, CD4 and CD8 (study of CD3+CD4+CD8-CD45RA-PD-1+CD38+ and CD3+CD4-CD8+CD45RA-PD-1+CD38+ subpopulations);
c) CD45RA, CD4, CD8 and CD127 (study of CD3+CD4+CD8-CD45RA-CD127-PD-1+ CD38+and CD3+CD4-CD8+CD45RA-CD127-PD-1+CD38+ subpopulations);
d) CD45RA, CD4, CD8, CD127 and CXCR5 (study of CD3+CD4+CD8-CD45RA-CD127-CXCR5-PD-1+ CD38+and CD3+CD4-CD8+CD45RA-CD127-CXCR5-PD-1+CD38+ subpopulations); and
e) CD45RA, CD4, CD8, CD127, CXCR5 and CD27 (study of CD3+CD4+CD8-CD45RA-CD127-CXCR5-CD27+PD-1+CD38+ and CD3+CD4-CD8+CD45RA-CD127-CXCR5-CD27+PD-1+CD38+ subpopulations)

Advantageously, the combinations of the mentioned markers can allow to reveal CD4 and CD8 T lymphocyte populations that are significantly increased in patients with an active autoimmune pathology, in particular an active AIH, in comparison to a reference value, or for example to patients with NASH when compared to AIH.

Another object of the invention relates to the use of the pool of PD-1 (Programmed cell death 1) and CD38 proteins as biological markers in a sample from a subject for the diagnosis, prediction and/or prognosis of an active autoimmune pathology in a subject.

Another object of the invention relates to a device for detecting the co-expression of PD-1 and CD38 at the surface of T lymphocytes in a sample taken from a subject, comprising:
(i) optionally means for obtaining a sample taken from the subject, and
(ii) means for detecting the co-expression of PD-1 and CD38 at the surface of T lymphocytes in said sample, and
(iii) means for determining the frequency of co-expression of PD-1 and CD38 in the sample. Advantageously, said means for determining the frequency of co-expression may allow to indicate whether this frequency in said sample is higher or lower than a reference value and/or whether this frequency in said sample differs or not from a reference value, said reference value representing a diagnosis, a prediction and/or a prognosis of an active autoimmune pathology in the subject.

Other advantages may also become apparent to the person skilled in the art on reading the examples below, illustrated by the annexed figures, given by way of illustration.

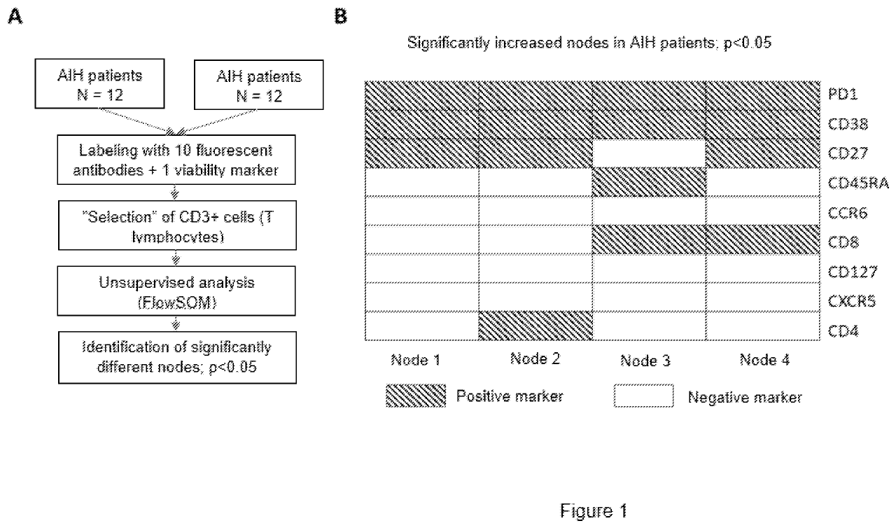

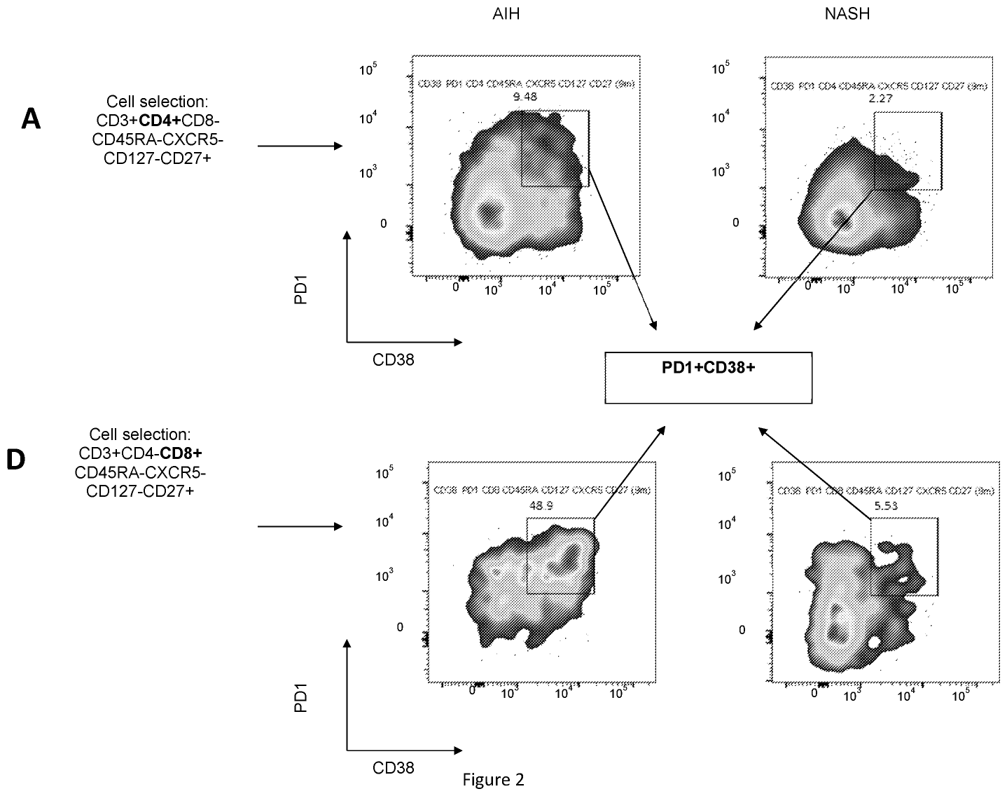

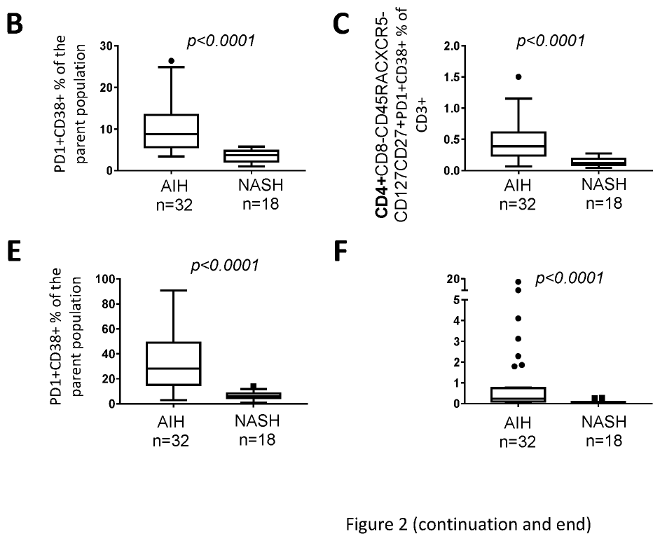

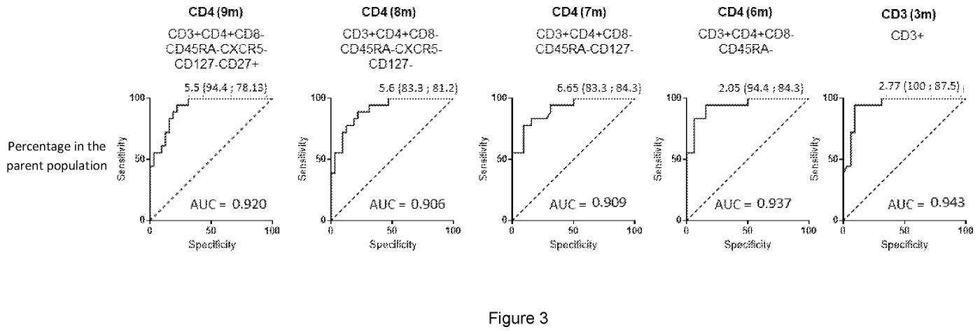

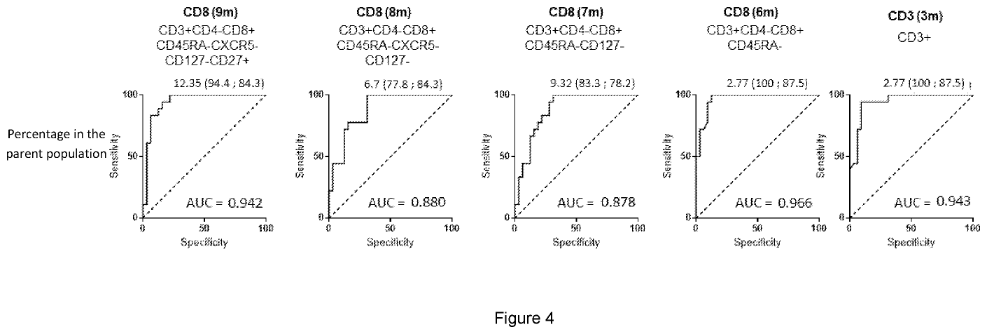

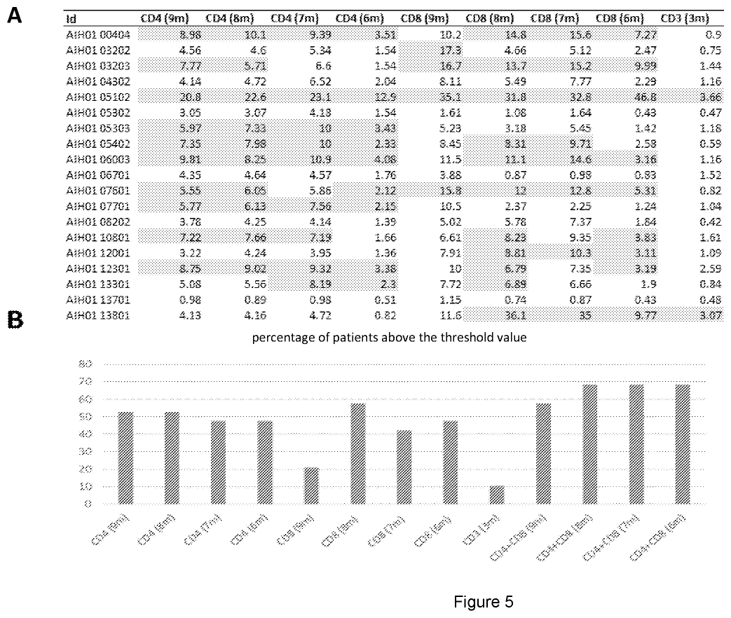

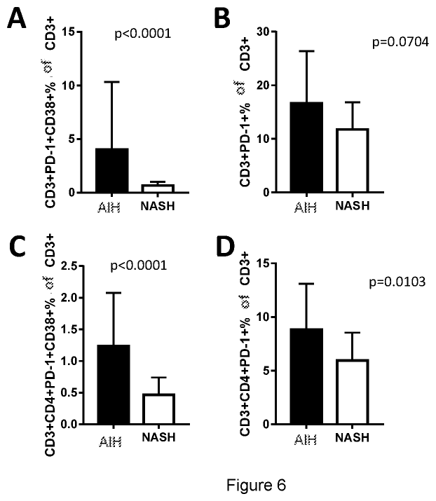

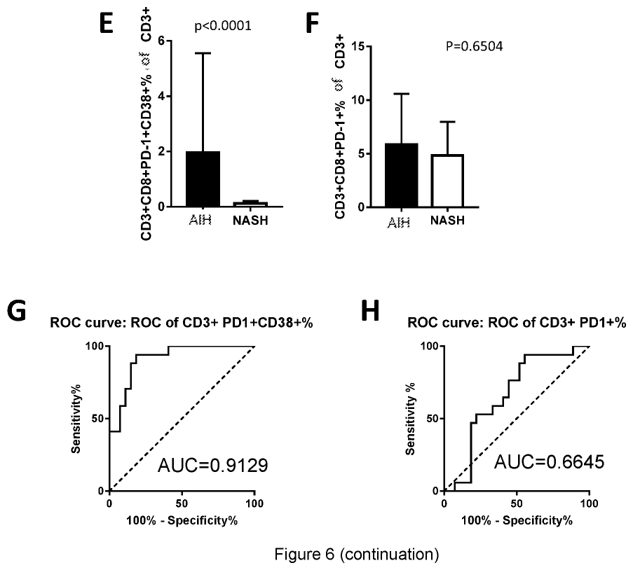

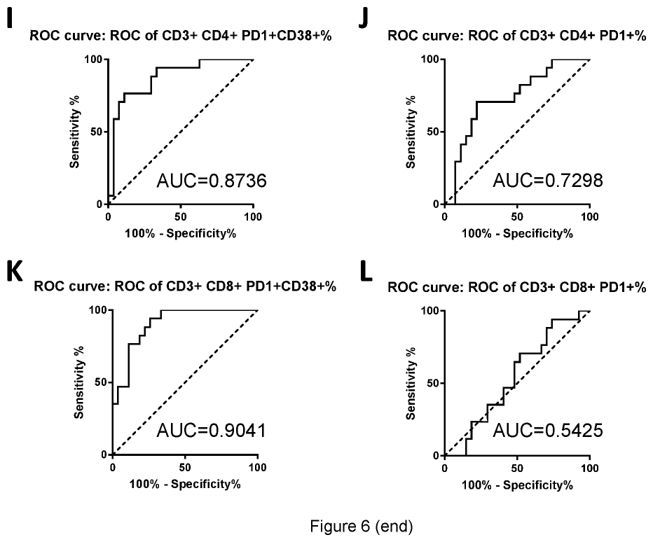

### BRIEF DESCRIPTION OF THE FIGURES

[FIG. 1] represents: A) representation of the unsupervised analysis strategy (FIowSOM) of flow cytometry data in 12 AIH and 12 NASH patients. B) Heatmap representing the expression of surface markers of the 4 lymphocyte populations (nodes) showing a significant increase (p<0.05) in AIH. Expression intensity ranging from striped (high expression) to white (low expression).

[FIG. 2] represents: A and D) Flow cytometry data representation of PD-1 and CD38 markers on CD3+CD4+CD45RA-CXCR5-CD127-CD27+(A) and CD3+CD8+CD45RA-CXCR5-CD127-CD27+(D) populations. B) and E) graphs representing the percentage of PD-1+CD38+cells per CD3+CD4+CD45RA-CXCR5-CD127-CD27+ cells (B) and per CD3+CD8+CD45RA-CXCR5-CD127-CD27+cells (E) in 32 AIH and 18 NASH patients. C and F) graphs representing the percentage of CD4+CD45RA-CXCR5-CD127-CD27+PD-1+CD38+(C) and CD8+CD45RA-CXCR5-CD127-CD27+PD1+CD38+(F) cells per CD3+cell. The p values were determined using the Mann-Whitney statistical test.

[FIG. 3] shows ROC curves based on the percentage of PD-1+CD38+cells of TLCD4 and TLCD3 in the parent population identified with the annotated markers above the ROC curve. In italics the cut-off value is shown in black with sensitivity and specificity values in parentheses. AUC values are shown on each graph.

[FIG. 4] shows ROC curves based on the percentage of PD-1+CD38+cells of TLCD8 and TLCD3 in the parent population identified with the annotated markers above the ROC curve. In italics the cut-off value is shown in black with sensitivity and specificity values in parenthesis. AUC values are shown on each graph.

[FIG. 5] is a representation of flow cytometry data values of PD-1 and CD38 markers in AIH patients in complete remission under treatment. A) Heatmap representing flow cytometry data values of PD-1 and CD38 markers in AIH patients in complete remission under treatment. In grey the data above the threshold values identified previously. B) Graphs representing the percentage of patients in complete remission under treatment with PD-1 and CD38 marker values above the previously determined threshold values (FIGS. 3 and 4).

[FIG. 6] is a representation of the analysis data between the simple measurement of PD-1 at the surface of T lymphocytes versus the measurement of co-expression of PD-1 with CD38 at the surface of T lymphocytes. Comparison of the percentage of CD3+PD-1+CD38+(A), CD3+PD-1+(B), CD3+CD4+PD-1+CD38+(C), CD3+CD4+PD-1+(D), CD3+CD8+PD-1+CD38+(E), and CD3+CD8+PD-1+(F) CD3+ cells between AIH and NASH patients. The p values were determined using the Mann-Whitney statistical test. ROC curves (% sensitivity/specificity) based on the percentage of CD3+PD-1+CD38+(G), CD3+PD-1+(H), CD3+CD4+PD-1+CD38+(I), CD3+CD4+PD-1+(J), CD3+CD8+PD-1+CD38+(K), and CD3+CD8+PD-1+(L) CD3+ cells between AIH and NASH patients are shown. AUC values are shown on each graph.

### EXAMPLES

### Example 1

### Identification of Differential Lymphocyte Populations Between AIH and NASH Patients

In order to identify a specific signature of hepatic autoimmunity, we performed analyses of multiparametric flow cytometry data by unsupervised statistical methods (FIowSOM) between 12 AIH patients and 12 NASH patients, which represent the best choice as control population to answer our question.

This unsupervised approach allowed us to establish a flow cytometric identification strategy, using a total of 13 parameters including 10 cell surface markers (CD3, CD4, CD8, CD45RA, CXCR5, CCR6, CD27, CD127, PD-1 and CD38) and 1 viability marker, of two cell populations (TL CD4 and CD8) that are characterized by PD-1 and CD38 expression (FIG. 1).

In a cohort of 32 active AIH patients and 18 NASH patients, the two cell populations TL CD4 and TL CD8 expressing PD-1 and CD38 are significantly increased in the blood of AIH patients compared to NASH patients (FIG. 2). This strategy allows easy discrimination between hepatic autoimmunity (AIH) and hepatic inflammation (ROC curve; FIGS. 3 and 4).

In comparison, the single measurement of PD-1 expression at the surface of T lymphocytes (CD3+, CD3+CD4+ and CD3+CD8+) is much less sensitive to discriminate between hepatic autoimmunity (AIH) and hepatic inflammation (NASH) than the measurement of PD-1 and CD38 co-expression at the surface of T lymphocytes (FIG. 6. ROC curve). Indeed, the analysis of the difference in the frequency of T lymphocytes expressing PD-1 between active AIH patients and NASH patients is much less significant than the results obtained with the measurement of PD-1 and CD38 co-expression (FIG. 6).

These results demonstrate the interest of using these markers, especially in flow cytometry, as potential biomarkers of hepatic autoimmunity.

### Example 2

### Determining Biomarker Cut-Off Values

In order to propose the most sensitive threshold values possible, we analyzed the generated results in various ways (FIGS. 3 and 4). The principle was to carry out the analysis of the percentage of PD-1 and CD38 expression by modifying the number of associated markers, in particular with a view to simplifying subsequent use.

In the CD4 TL (FIG. 3) and CD8 TL (FIG. 4) populations, and in the total CD3 TL population (FIGS. 3 and 4), the percentage of PD-1 and CD38 expression (in the parent cell population according to a “gating” strategy for the analysis of flow cytometry results) was analyzed in the CD45RA−(6m); CD45RA-CD127−(7m); CD45RA-CD127-CXCR5−(8m) and CD45RA-CD127-CXCR5-CD27+(9m) subpopulations (FIGS. 3 and 4). The analysis was performed using ROC curves. In all conditions of analysis we obtain areas under the curve (AUC) greater than 0.85, demonstrating the strong potential of these markers as biomarkers of hepatic autoimmunity.

Threshold values for the autoimmune signature could thus be defined with sensitivities ranging from 77% to 100% and specificities ranging from 78% to 87%.

In a small group of AIH patients in complete remission under treatment (pilot study), we observed that about 50% of AIH patients in complete remission under treatment (n=19) maintained an elevated level of these biomarkers, whereas the level was normalized in the others (FIG. 5). This suggests a possible association between the persistence of these blood biomarkers and patient outcome (risk of relapse).

All these results demonstrate that it is possible to define an autoimmune lymphocyte signature in the blood of patients that is discriminative of non-specific inflammation.

### Example 3

### Searching for Combinations of Biomarkers of Interest

The study focuses on 1) the clinical interpretation of the cytometry data; 2) the validation of the data in a larger number of patients and in other autoimmune pathologies; and 3) the simplification and development of a test prototype on whole blood.

Concerning the simplification and development of a test prototype on whole blood, different associations of grouping of the previously mentioned markers are tested, in order to group the negative markers together to create “windows” of exclusions and thus facilitate the reading of the test.

For example, for the 9m panel, an initial selection of CD3+CD45RA-CXCR5-CD127-T cells is made, with CD45RA, CXCR5 and CD127 grouped in the same reading channel to facilitate their exclusion. It is thus possible to go from 9 reading channels in flow cytometry to 6 or 7 channels.

This makes it possible to find the best possible combinations to offer a simple and most sensitive test.

A sensitivity study is further performed on the analysis of the co-expression of PD-1 and CD38 at the surface of CD3 TL (CD3+) in association with the marker(s): (a) CD45RA (study of the CD3+CD45RA-PD-1+CD38+subpopulation); (b) CD45RA, CD4 and CD8 (study of the CD3+CD4+CD8-CD45RA-PD-1+CD38+and CD3+CD4-CD8+CD45RA-PD-1+CD38+ subpopulations); c) CD45RA, CD4, CD8 and CD127 (study of CD3+CD4+CD8-CD45RA-CD127-PD-1+CD38+ and CD3+CD4-CD8+CD45RA-CD127-PD-1+CD38+ subpopulations); d) CD45RA, CD4, CD8, CD127 and CXCR5 (study of CD3+CD4+CD8-CD45RA-CD127-CXCR5-PD-1+CD38+and CD3+CD4-CD8+CD45RA-CD127-CXCR5-PD-1+CD38+ subpopulations); and e) CD45RA, CD4, CD8, CD127, CXCR5 and CD27 (study of the CD3+CD4+CD8-CD45RA-CD127-CXCR5-CD27+PD-1+CD38+and CD3+CD4-CD8+CD45RA-CD127-CXCR5-CD27+PD-1+CD38+ subpopulations)

The purpose of this study is to identify the most sensitive association of marker associated with the co-expression of PD-1 and CD38 to measure autoimmune activity in patients with autoimmune hepatitis.

### LIST OF REFERENCES

1. Liberal, R., Krawitt, E. L., Vierling, J. M., Manns, M. P., Mieli-Vergani, G., and Vergani, D. (2016). Cutting edge issues in autoimmune hepatitis. J. Autoimmun. 75, 6-19.

2. Manns, M. P., Czaja, A. J., Gorham, J. D., Krawitt, E. I., Mieli-Vergani, G., Vergani, D., Vierling, J. M., and American Association for the Study of Liver Diseases (2010). Diagnosis and management of autoimmune hepatitis. Hepatol. Baltim. Md 51, 2193-2213.

3. European Association for the Study of the Liver (2015). EASL Clinical Practice Guidelines: Autoimmune hepatitis. J. Hepatol. 63, 971-1004.

4. Renand, A., Habes, S., Mosnier, J.-F., Aublé, H., Judor, J.-P., Vince, N., Hulin, P., Nedellec, S., Métairie, S., Archambeaud, I., et al. (2018). Immune Alterations in Patients With Type 1 Autoimmune Hepatitis Persist Upon Standard Immunosuppressive Treatment. Hepatol. Commun. 2, 968-981.

5. Böttcher, K., Rombouts, K., Saffioti, F., Roccarina, D., Rosselli, M., Hall, A., Luong, T., Tsochatzis, E. A., Thorburn, D., and Pinzani, M. (2018). MAIT cells are chronically activated in patients with autoimmune liver disease and promote pro-fibrogenic hepatic stellate cell activation. Hepatol. Baltim. Md.

6. Jeffery, H. C., van Wilgenburg, B., Kurioka, A., Parekh, K., Stirling, K., Roberts, S., Dutton, E. E., Hunter, S., Geh, D., Braitch, M. K., et al. (2016). Biliary epithelium and liver B cells exposed to bacteria activate intrahepatic MAIT cells through MRI. J. Hepatol. 64, 1118-1127.

7. Oo, Y. H., Banz, V., Kavanagh, O., Liaskou, E., Withers, D. R., Humphreys, E., Reynolds, G. M., Lee-Turner, L., Kalia, N., Hubscher, S. G., et al. (2012). CXCR3-dependent recruitment and CCR6-mediated positioning of Th-17 cells in the inflamed liver. J. Hepatol. 57, 1044-1051.

1. A method for diagnosis, prediction and/or prognosis of an active autoimmune pathology in a subject, comprising detecting the co-expression of the proteins PD-1 (Programmed cell death 1) and CD38 at the surface of T lymphocytes in a biological sample of the subject.

2. The method according to claim 1, wherein said biological sample is a blood fraction or a whole blood sample, or a biopsy fraction, for example liver, of tissue.

3. The method according to claim 1, wherein the proteins PD-1 and CD38 are detected at the surface of CD3 and/or CD8 T lymphocytes and/or CD4 T lymphocytes.

4. The method according to claim 1, further comprising the steps of:
(i) detecting the co-expression, at the surface of T lymphocytes, of PD-1 and CD38 in said sample and determining the frequency of co-expression of PD-1 and CD38;
(ii) comparing the frequency of co-expression of PD-1 and CD38 determined in (i) with a reference value of the frequency of co-expression of PD-1 and CD38;
(iii) testing for the presence or absence of a deviation of the frequency of co-expression of PD-1 and CD38 determined in (ii) from the reference value; and
(iv) attributing the presence or absence of a deviation to a diagnosis, prediction and/or prognosis of the subject's active autoimmune pathology.

5. The method according to claim 1, wherein a frequency of co-expression of PD-1 and CD38 in said sample above a reference value indicates that the subject has the active autoimmune pathology or presents a risk of relapse of the active autoimmune pathology.

6. The method according to claim 1, wherein expression of at least one other biomarker selected from CD3, CD4, CD8, CD45RA, CXCR5, CD127 and/or CD27 is detected.

7. The method according to claim 6, wherein co-expression of biomarker associations selected from PD-1/CD38/CD3, PD-1/CD38/CD3/CD4/CD8/CD45RA,
PD-1/CD38/CD3/CD4/CD8/CD45RA/CD127,
PD-1/CD38/CD3/CD4/CD8/CD45RA/CXCR5/CD127, and/or
PD-1/CD38/CD3/CD4/CD8/CD45RA/CXCR5/CD127/CD27 associations are detected.

8. The method according to claim 6, wherein the frequency of lymphocyte populations defined by the following phenotypes is detected:
a) CD3+CD45RA-PD-1+CD38+;
b) CD3+CD4+CD8-CD45RA-PD-1+CD38+and/or CD3+CD4-CD8+CD45RA-PD-1+CD38+;
c) CD3+CD4+CD8-CD45RA-CD127-PD-1+CD38+and/or CD3+CD4-CD8+CD45RA-CD127-PD-1+CD38+;
d) CD3+CD4+CD8-CD45RA-CD127-CXCR5-PD-1+CD38+and/or CD3+CD4-CD8+CD45RA-CD127-CXCR5-PD-1+CD38+; and/or
e) CD3+CD4+CD8-CD45RA-CD127-CXCR5-CD27+PD-1+CD38+and/or CD3+CD4-CD8+CD45RA-CD127-CXCR5-CD27+PD-1+CD38+.

9. The method according to claim 1, wherein the detection is performed by a method selected from flow or mass cytometry, an immunoassay technology, such as direct ELISA, indirect ELISA, sandwich ELISA, competitive ELISA, multiplex ELISA, radioimmunoassay (RIA) or ELISPOT technology, a mass spectrometry analysis method, a chromatography method, a qPCR method, and a combination of at least two of these methods.

10. The method according to claim 1, wherein said prediction is a prediction of the risk of relapse, in particular following the cessation or adjustment of a treatment.

11. The method according to claim 1, wherein said prediction is a prediction of the risk of developing an autoimmune pathology.

12. The method according to claim 1, wherein said active autoimmune pathology is autoimmune hepatitis, primary biliary cholangitis, primary sclerosing cholangitis, type 1 diabetes, multiple sclerosis, rheumatoid arthritis, lupus, or vasculitis or immunological toxicity related to immunotherapies in cancer treatment.

13. The method according to claim 1, wherein said active autoimmune pathology is autoimmune hepatitis.

14. The method according to claim 13, wherein said autoimmune hepatitis is an atypical form autoimmune hepatitis, including a seronegative form and/or with little or no lymphocytic infiltrate of the liver tissue.

15. The method according to claim 13, wherein the diagnosis is a discrimination of patients with autoimmune hepatitis from patients with non-alcoholic steatohepatisis (NASH), or a demonstration of hepatic autoimmunity in patients with NASH.

16. (canceled)

17. A test device for detecting the co-expression of PD-1 and CD38 at the surface of T lymphocytes in a sample from a subject, comprising:
(i) optionally means for obtaining a sample from the subject, and
(ii) means for detecting the co-expression of PD-1 and CD38 at the surface of T lymphocytes in said sample, and
(iii) means for determining the frequency of co-expression of PD-1 and CD38 in the sample.
